(19) United States
(12) Patent Application Publication
(10) Pub. No.: US 2021/0196855 A1
(43) Pub. Date: Jul. 1, 2021

(54) COMPOSITIONS AND USES OF NANOSCALE DIAMOND PARTICLES FOR ARTIFICIAL JOINT

(71) Applicant: UNIVERSITY OF NORTH TEXAS, DENTON, TX (US)

(72) Inventors: DIANA BERMAN, Denton, TX (US); DONGHUI ZHU, Frisco, TX (US); OLGA SHENDEROVA, Raleigh, NC (US)

(73) Assignee: ADAMAS NANOTECHNOLOGIES, INC., RALEIGH, NC (US)

(21) Appl. No.: 17/185,513

(22) Filed: Feb. 25, 2021

Related U.S. Application Data
(63) Continuation-in-part of application No. 17/015,246, filed on Sep. 9, 2020.
(60) Provisional application No. 62/898,323, filed on Sep. 10, 2019.

(51) Int. Cl. A61L 27/08; A61L 27/06; A61L 27/20; A61L 27/54; A61L 27/18

(52) U.S. Cl. CPC A61L 27/08; A61L 27/06; A61L 27/20; A61L 27/54; A61L 27/18; A61L 2430/24; A61L 2400/12; A61L 2400/06; A61L 2300/41; A61L 2300/404; A61L 2300/452

(57) ABSTRACT

A new insight on the lubrication of joints is presented. Addition of small amounts of nanoscale diamond particles to a joint promotes a substantial improvement in friction and wear behavior of the joint surfaces. The joints can be artificial or natural joints.

### CLAIM OF PRIORITY TO RELATED APPLICATION

This application claims priority to co-pending U.S. non-provisional application entitled “COMPOSITIONS AND USES OF NANOSCALE DIAMOND PARTICLES FOR JOINTs” having Ser. No. 17/015,246 filed on Sep. 9, 2020, which claims the benefit of U.S. Provisional Application Ser. No. 62/898,323, filed Sep. 10, 2019, both of which are incorporated herein by reference in their entirety.

### BACKGROUND

Premature natural joint degeneration is a common problem in the population over the age of 40 as a result of excessive loading conditions as well as failure of normal repair processes. One natural joint problem is osteoarthritis (OA), which is a chronic degenerative joint disease that is the result of a combination of problems: degeneration of articular cartilage, lubrication deficiency, synovium inflammation. Various approaches to treating OA are known, but they have one or more deficiencies.

Early-stage OA is commonly manifested by the degeneration of articular cartilage concomitant with lubrication deficiency and synovium inflammation, presenting a complex interaction of mechanical, biochemical, and cellular processes within the joint. Impaired lubrication leads to weakened and overloaded cartilage and adverse cellular responses which, in turn, further compromise lubrication. Specifically, due to inferior lubrication, the production of degradative enzymes by the chondrocytes and the inflamed synovium is greatly enhanced, resulting in collagen digestion in the cartilage and reduction in its mechanical properties. Weakened cartilage is unable to withstand the mechanical loading which further increases wear and damage. Existing treatment approaches include oral medication, intra-articular (IA) injection of lubricating fluids, or interventional micro-fracturing to repair local cartilage defects. Oral administration of anti-inflammatory or chondroprotective drugs is accompanied by multiple systemic side effects and adverse consequences from interactions with other medications commonly prescribed for elderly patients. Current practice is intra-articular injections of hyaluronic acid as a basic component replenishing the lubricating fluid in the joint. The injections are usually administered in a series of ˜3-4 injections over a 6-month period, thus highlighting their limited effectiveness. Corticosteroids administered by injection at the same time or in parallel are usually used to relieve pain and reduce swelling; if used repeatedly, they also produce side effects. Therefore, there is a critical need for new approaches that improve the effectiveness and safety of the early OA treatment. New strategies include intra-articular injectable nanocarriers (polymer, liposome) to accomplish prolonged lubrication accompanied by sustained local drug release. However the underlying problem of structurally weakened cartilage remains unsolved. Collagen crosslinking treatments are being developed to increase cartilage stiffness, however this approach involves surgical intervention.

Another long-term approach is the use of artificial joints, which are made from metal, ceramic, or plastic materials and have become the only long-term solution for relief from pain, mobility, or other adverse health effects related to joint degradation and failure. In recent years, the number of orthopaedic surgeries substantially increased, though reliability and lifetime of the artificial joints remain a major issue.

During operation, artificial joints are exposed to a complex environment and subjected to mechanical degradation. Additionally, biocompatibility of the materials, or their ability not to cause an inflammatory or toxic response, is an important aspect to consider.

The search for biocompatible, tribologically efficient materials led to the exploration of different ceramic and metal alloy components. Ideally, the joint replacement material should exhibit an identical performance to the bone when in operation. Ultra-high-molecular-weight polyethylene (UHMWPE) was used in earlier years, but raised concerns with regard to adverse tissue reactions. UHMWPE was replaced with stainless steel and then with Co—Cr—Mo and alumina, which demonstrated good wear resistance but lead to inflammation and pain in long-term. So far, titanium remains the most favorable materials for artificial joints. This has led to extensive research on titanium-based alloys for biomedical applications, such as Ti—Nb—Ta—Zr or TNZT, Ti-6A-7Nb, Ti-6Al-4V, and Ti-5Al-2.5Fe, among others. Titanium and titanium-based alloys are the preferred materials used for hip cup shells due to their high corrosion resistance and biocompatibility over other materials, such as conventional stainless steels and cobalt-based alloys. However, high wear of the titanium components during exposure to normal and shear stresses is a major cause for their failure. As a result, degradation of the metal implants during movement of the joints limits their lifetime.

### SUMMARY

In an aspect of the present disclosure, nanoscale diamond particles (NDs) have proven to be excellent friction and wear modifiers in various sliding systems. Specifically, adding small amounts of nanoscale diamond particles to joints (e.g., natural or artificial joints) resulted in substantial decrease in friction and wear of joint surfaces. NDs can provide lubrication for joints. NDs are also used for reinforcement of polymers and hydrogels. Polymer nanocomposites reinforced with NDs demonstrate significant improvement in resistance to wear, decreased coefficient of friction, and improved durability in tribological experiments. Upon incorporation into a cartilage, NDs are expected to provide strengthening of a cartilage. Therefore, combinatorial approach simultaneously providing structural reinforcement of the weakened cartilage and enhanced lubricity using the same nanotherapeutic, the NDs, is expected to be a more efficient treatment than addressing the underlaying biomechanical deficiencies separately. In addition, NDs have been demonstrated as very efficient carriers of therapeutics and have shown markedly improved efficacy and safety for treatment in multiple preclinical studies. The loading capacity and binding strength of drug molecules adsorbed/covalently grafted on NDs can be well-controlled through engineering of surface groups. Therefore, it is expected that NDs can also play a role of delivery of drugs for treatment of OA such as, for example, anti-inflammatory or chondroprotective drugs. In terms of biocompatibility, different forms of carbon were already considered in various in-vivo and in-vitro studies. Previous studies demonstrated biocompatibility of nanoscale diamond particles. Naturally occurring graphitic layers have been detected in metal-on-metal hip replacements. Therefore, nanoscale diamond particles are an ideal solution for improving the longevity of artificial joint components in the body.

Suitable methods of applying nanoscale diamond particles to a joint include (i) coating an effective amount of nanoscale diamond particles onto the artificial joint prior to implant; (ii) applying a composition to the artificial joint during an artificial joint implant surgery, where said composition comprises a biocompatible carrier fluid and an effective amount of nanoscale diamond particles dispersed in the biocompatible carrier fluid; (iii) injecting the composition into the artificial joint or natural joint. In certain embodiments, the metered formulation of NDs in biocompatible media for intraarticular injections can comprise a therapeutically effective amount of hyaluronic acid (HA), hyaluronate salt, derivatives of HA, or other viscosupplements. In yet another embodiment NDs can delivery drugs into joint fluids and cartilage.

In an aspect, the present disclosure provides for a method of lubricating a joint in a subject in need thereof, the method comprising: introducing an effective amount of a composition into the joint to lubricate the joint, wherein the joint is an artificial joint or a natural joint, wherein the composition comprises (i) a biocompatible carrier fluid; and (ii) an effective amount of nanoscale diamond particles dispersed in the biocompatible carrier fluid to lubricate the joint when applied to the joint in the subject. The intraarticular injection can also comprise therapeutically effective amount hyaluronic acid, hyaluronate salt, derivatives of hyaluronic acid, or other viscosupplements and drugs such as anti-inflammatory drugs, antibacterial drugs, corticosteroids, chondroprotective drugs or other drugs used for the treatment of OA.

In an aspect, the present disclosure provides for a method of lubricating a joint in a human, the method comprising: injecting an effective amount of a composition into the joint to lubricate the joint, wherein the joint is a natural joint, wherein the composition comprises (i) a biocompatible carrier fluid that is selected from the group consisting of a simulated body fluid, a synovial fluid, a combination thereof, and mixtures thereof with one or more additional fluids; and (ii) an effective amount of nanoscale diamond particles dispersed in the biocompatible carrier fluid to lubricate the joint when applied to the joint in the subject, wherein the nanoscale diamond particles are present at a concentration of about 0.1 wt % to about 0.15 wt % based upon an entire weight of the composition, wherein the nanoscale diamond particles comprise carbon atoms, and wherein the carbon atoms have an orbital hybridization that is about 80% to about 99% sp³ carbon, and wherein the nanoscale diamond particles are spherical and have a volumetric size of about 2 nm to about 6 nm. Injecting can include an intra-articular injection into a hip joint, a joint of hand, an elbow joint, a wrist joint, a glenohumeral joint, an acromioclavicular joint, a sternoclavicular joint, a vertebral articulation, a temporomandibular joint, a sacroiliac joint, a knee joint, and an articulation of foot.

In an aspect, the present disclosure provides for a composition for lubricating a joint in a subject in need thereof; the composition comprising: (i) a biocompatible carrier fluid; and (ii) an effective amount of nanoscale diamond particles dispersed in the biocompatible carrier fluid to lubricate the joint when applied to the joint in the subject. The joint can be a natural joint or an artificial joint. The joint, artificial or natural, can include: a hip joint, a joint of hand, an elbow joint, a wrist joint, a glenohumeral joint, an acromioclavicular joint, a sternoclavicular joint, a vertebral articulation, a temporomandibular joint, a sacroiliac joint, a knee joint, or an articulation of foot.

In an aspect, the present disclosure provides for a method of lubricating a joint in a subject in need thereof, the method comprising: coating an effective amount of nanoscale diamond particles onto the joint to lubricate the joint and/or applying an effective amount of a composition as described above and herein, wherein the joint is an artificial joint or a natural joint.

### BRIEF DESCRIPTION OF THE DRAWINGS

Further aspects of the present disclosure will be readily appreciated upon review of the detailed description of its various aspects, described below, when taken in conjunction with the accompanying drawings.

FIG. 1A is a graph of the coefficient of friction values during sliding at 0.25 N load in different concentrations of ND solution in simulated body fluid (SBF).

FIG. 1B is a picture of the wear tracks formed during sliding at 0.25 N load in different concentrations of ND solution in SBF.

FIG. 2A is a graph of the coefficient of friction values during sliding in different concentration of ND in SBF under 0.25 N applied load.

FIG. 2B is a graph of the coefficient of friction values during sliding in different concentration of ND in SBF under 0.5 N applied load.

FIG. 2C is a graph of the coefficient of friction values during sliding in different concentration of ND in SBF under 1 N applied load.

FIG. 3A is a graph of the wear of the titanium surfaces during the tests at different loads. At 0.25 N load (Hertzian contact pressure 0.28 GPa), the minimum wear is observed for 0.05 wt % of NDs; at 0.5 N load (Hertzian contact pressure 0.35 GPa), the minimum wear is for 0.1 wt. % NDs; and at 1 N load (Hertzian contact pressure 0.45 GPa), the minimum wear is for 0.15 wt. % NDs.

FIG. 3B is optical images of the ball wear scar for the pure SBF and SBF with optimal concentrations of NDs.

FIG. 4A is a SEM-EDS analysis of the wear track formed during the tests in SBF at 0.5 N load (Hertzian contact pressure 0.35 GPa). Analysis indicates a high concentration of oxygen inside the wear track. The Na, F, and Cl peaks originate from the residue of the SBF on the surface and are intentionally excluded from the further analysis of the oxidation amount.

FIG. 4B is a SEM-EDS analysis of the wear track formed during the tests in SBF+0.1 wt. % NDs at 0.5 N load (Hertzian contact pressure 0.35 GPa). Inside the wear track, the presence of carbon is observed. The Na, F, and C peaks originate from the residue of the SBF on the surface and are intentionally excluded from the further analysis of the oxidation amount.

FIGS. 5A-5F are a SEM-EDS map of the wear track formed during the test in SBF+0.1 wt. % NDs at 0.5 N load (Hertzian contact pressure 0.35 GPa). (FIG. 5A) The area of the wear track with (FIG. 5B) higher magnification image is highlighted. (FIG. 5C) Overlay map and detailed (FIG. 5D) carbon, (FIG. 5E) oxygen and (FIG. 5F) titanium map demonstrate uniformity of the titanium and oxygen concentration inside and outside wear track while carbon is found inside the wear track only.

FIGS. 6A-6D are a Raman 2D map of the sp³ bonded carbon peak (at ˜1330 cm-1) of the wear track formed during the test with SBF+0.1 wt. % NDs at 0.5 N load (Hertzian contact pressure 0.35 GPa). (FIG. 6A) Overview of the wear track selected for the Raman 2D map analysis. (FIG. 6B) Raman spectra inside and outside the wear track for the tests performed in (FIG. 6C) SBF+0.1 wt. % NDs and (FIG. 6C) pure SBF.

FIG. 7A is pictures of colony formation assay of S. aureus treated with ND.

FIG. 7B is a graph of colony formation assay of S. aureus treated with ND.

FIGS. 8A-8D display the antibacterial activity of ND on E. coli. Bacteria growth curve treated with different concentration of ND (FIG. 8A) and ND-BSA (FIG. 8B); (FIG. 8C) E. coli growth and adhesion on Ti and Ti-ND plates by SEM; (FIG. 8D) Comparison of numbers of E. coli attached to Ti and Ti-ND plates; * indicates p<0.05, * * indicates p<0.01, * * * indicates p <0.001.

FIG. 9 is a graph of the effects of NDs on cell viability. Effects of ND-BA on cell viability. MC3T3-E1 cells were treated with different concentration of NDs and incubated for 3 days after treatment. 3-(4,5-dimethylthiazol2-yl)-2,5-diphenyltetrazolium bromide) (MTT) was performed to measure cell viability.

FIG. 10 is a graph of the optical density (OD) measurements of bacteria at a wavelength of 600 nm (OD 600 nm) for S. aureus. S. aureus was incubated with different concentration of NDs for 16 hours and bacteria growth was measured at OD 600 nm.

FIG. 11 illustrates schematics of a synergistic approach using NDs for treatment of early-stage OA and wear-induced damage in the joints.

FIG. 12 illustrates graphs showing the friction (a) and wear (b,c) reduction in synovial fluid with NDs in contrast to pure SF in reciprocating sliding test on polymeric HDPE substrate at different ND dosing and load. Smoothened porcine cartilage (d) with negligible wear lubricated with NDs in saline in a prolonged test (100,000 cycles, 1 Hz, 0.5N load).

### DETAILED DESCRIPTION

Before the present disclosure is described in greater detail, it is to be understood that this disclosure is not limited to particular aspects described, and as such may, of course, vary. It is also to be understood that the terminology used herein is for the purpose of describing particular aspects only, and is not intended to be limiting. The skilled artisan will recognize many variants and adaptations of the aspects described herein. These variants and adaptations are intended to be included in the teachings of this disclosure.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant specification should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

Although any methods and materials similar or equivalent to those described herein can also be used in the practice or testing of the present disclosure, the preferred methods and materials are now described. Functions or constructions well-known in the art may not be described in detail for brevity and/or clarity. Aspects of the present disclosure will employ, unless otherwise indicated, techniques of nanotechnology, organic chemistry, material science and engineering and the like, which are within the skill of the art. Such techniques are explained fully in the literature.

It should be noted that ratios, concentrations, amounts, and other numerical data can be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to about 5%” should be interpreted to include not only the explicitly recited values of about 0.1% to about 5%, but also include individual values (e.g., 1%, 2%, 3%, and 4%) and the sub-ranges (e.g., 0.5%, 1.1%, 2.2%, 3.3%, and 4.4%) within the indicated range. Where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In some aspects, the term “about” can include traditional rounding according to significant figures of the numerical value. In addition, the phrase “about ‘x’ to ‘y”’, where ‘x’ and ‘y’ are numerical values, includes “about ‘x’ to about ‘y”’.

In some instances, units may be used herein that are non-metric or non-SI units. Such units may be, for instance, in U.S. Customary Measures, e.g., as set forth by the National Institute of Standards and Technology, Department of Commerce, United States of America in publications such as NIST HB 44, NIST HB 133, NIST SP 811, NIST SP 1038, NBS Miscellaneous Publication 214, and the like. The units in U.S. Customary Measures are understood to include equivalent dimensions in metric and other units (e.g., a dimension disclosed as “1 inch” is intended to mean an equivalent dimension of “2.5 cm”; a unit disclosed as “1 pcf” is intended to mean an equivalent dimension of 0.157 kN/m³; or a unit disclosed 100° F. is intended to mean an equivalent dimension of 37.8° C.; and the like) as understood by a person of ordinary skill in the art.

### Definitions

Unless defined otherwise, all technical and scientific terms used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

The articles “a” and “an,” as used herein, mean one or more when applied to any feature in aspects of the present invention described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used.

The term “volumetric size”, as used herein, refers to the size of the nanoscale diamond particles. In practice, the volumetric size of nanoscale diamond particles can be estimated or characterized by dynamic light scattering. DLS volumetric sizes are reported as a % composition of the total volume with particles having a given range of diameters.

The term “coefficient of friction”, as used herein, refers to a value that corresponds to the relationship between friction forces of two objects. The friction force is a force exerted by a surface when an object moves across it, or makes an effort to move across it.

The term “biocompatible”, as used herein, may refer to the ability of the material to perform its intended function, with the desired degree of incorporation in the host, without eliciting any undesirable local or systemic effects in that host.

The term “hertzian contact pressure”, as used herein, refers to the localized stress pressure that develops as two curved surfaces come in contact and deform slightly under imposed loads. This amount of deformation is dependent on the modulus of elasticity of the materials in contact. It gives the contact stress as a function of the normal contact force, the radii of curvature of both bodies and the modulus of elasticity of both bodies. Hertzian contact stress pressure forms the foundation for the equations for load bearing capabilities and fatigue life in bearings, gears, and any other bodies where two surfaces are in contact.

As used herein, “about,” “approximately,” and the like, when used in connection with a numerical variable, generally refers to the value of the variable and to all values of the variable that are within the experimental error (e.g., within the 95% confidence interval for the mean) or within +/−10% of the indicated value, whichever is greater.

As used herein, “subject” refers to any living entity comprised of at least one cell. A living organism can be as simple as, for example, a single isolated eukaryotic cell or cultured cell or cell line, or as complex as a mammal, including a human being, and animals (e.g., vertebrates, amphibians, fish, mammals, e.g., cats, dogs, horses, pigs, cows, sheep, rodents, rabbits, squirrels, bears, primates (e.g., chimpanzees, gorillas, and humans). In particular, living organism is a human or horse or household pet.

The term “therapeutically effective amount” refers to the amount of the composition used is of sufficient quantity to ameliorate one or more causes or symptoms of a disease or disorder, such as OA. Such amelioration only requires a reduction or alteration, not necessarily elimination.

The term “treatment” refers to the medical management of a patient with the intent to cure, ameliorate, stabilize, or prevent a disease, pathological condition, or disorder, in particular OA. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder, such as OA; preventative treatment, that is, treatment directed to minimizing or partially or completely inhibiting the development of the associated disease, pathological condition, or disorder, such as OA; and supportive treatment, that is, treatment employed to supplement another specific therapy directed toward the improvement of the associated disease, pathological condition, or disorder, such as OA.

Reference throughout this specification to “one embodiment”, “an embodiment”, “another embodiment”, “some embodiment,” means that a particular feature, structure or characteristic described in connection with the embodiment is included in at least one embodiment of the present invention. Thus, appearances of the phrases “in one embodiment,” “in an embodiment,” “in another embodiment”, or “in some embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

### DISCUSSION

In some aspects, the present disclosure provides a composition for lubricating a joint containing (i) a biocompatible carrier fluid; and (ii) an effective amount of nanoscale diamond particles dispersed in the biocompatible carrier fluid to reduce friction at the joint (e.g., natural or artificial); and decreased rate of cartilage deterioration. In another embodiment the composition also comprises a therapeutic amount of hyaluronic acid, hyaluronate salt, derivatives of HA, or other viscosupplement. In yet another embodiment NDs carry drug molecules in adsorbed or conjugated composition.

Nanoscale diamond particles are used for friction and wear reduction in artificial joint implants or in natural joints. Addition of small amounts of nanoscale diamond particles to a biocompatible carrier fluid has a substantial reduction in friction and wear of the surfaces.

In one or more aspects, the nanoscale diamond particles on its own, as well as when dissolved or dispersed in a biocompatible carrier fluid, are effective to reduce coefficient of friction by at least 20% (e.g., at least 30%, at least 40%, at least 50%, 60%, at least 75%, at least 90%, at least 95% (e.g., about 20% to 50%, about 20% to 75%, about 30% to 50%, about 30% to 75%, about 40% to about 75%, about 50 to 100%, about 50 to 90%, about 60 to 90%, about 70 to 90%, about 80 to 90%, about 80 to 95%)) as compared to the otherwise same composition except without the nanoscale diamond particles.

In one or more aspects, the nanoscale diamond particles on its own, as well as when dissolved or dispersed in a biocompatible carrier fluid, are effective to reduce wear by at least 20% (e.g., at least 30%, at least 40%, at least 50%, 60%, at least 75%, at least 90%, at least 95% (e.g., about 20% to 50%, about 20% to 75%, about 30% to 50%, about 30% to 75%, about 40% to about 75%, about 50 to 100%, about 50 to 90%, about 60 to 90%, about 70 to 90%, about 80 to 90%, about 80 to 95%)) as compared to the otherwise same composition except without the nanoscale diamond particles.

In one or more aspects, the nanoscale diamond particles on its own, as well as when dissolved or dispersed in a biocompatible carrier fluid, are effective to reduce bacteria growth by at least 20% (e.g., at least about 30%, at least about 45%, about least about 60%, at least about 75%, about least about 90% (e.g., about 20 to 90%, about 20 to 75%, about 45 to 90%, about 60 to 90%, about 75 to 90%)) as compared to the otherwise same composition except without the nanoscale diamond particles.

In one embodiment, the physical shape of the nanoscale diamond particles can be spherical, elliptical, faceted, or a mixture thereof. In another embodiment, the nanoscale diamond particles have a volumetric size of about 1 nm to about 20 nm, about 1 nm to about 10 nm, about 2 nm to about 10 nm, about 2 nm to about 8 nm, about 2 nm to about 6 nm, about 3 nm to about 5 nm, about 3 nm, about 4 nm, about 5 nm, about 6 nm, about 7 nm, about 9 nm or about 10 nm. In another embodiment, nanoscale diamond particles can form tight aggregates (unbreakable by ultrasonic treatment), where the tight aggregates have a volumetric size of about 10 nm to about 50 nm.

The nanoscale diamond particles may have the surfaces chemically modified (e.g., functionalized) for example carboxylated, which is referred to the surface-modification of nanoscale diamond particles with carboxylate functionality. This functionality can be achieved by oxidation reaction of nanoscale diamond particles with acids such as sulfuric acid and nitric acid. Carboxylated, hydroxilated, aminated, fluorinated, hydrogenated, NDs with silane, acrylic groups, aliphatic chains and other functionalities were produced. One embodiment includes diamond particles functionalized with poly(glycerol) by ring opening polymerization in neat glycidol to introduce colloidal stability in biological media followed by conjugation to therapeutic molecules by carbodiimide or related activation. Additionally, biological compatible excipients can be used to stabilize the colloid including serum albumin. In an aspect, the nanoscale diamond particles can be functionalized to include an agent such as a drug (e.g., a therapeutic amount of hyaluronic acid, hyaluronate salt, derivatives of HA, or other viscosupplement or other agents such as anti-inflammatory drugs, antibacterial drugs, corticosteroids, chondroprotective drugs or other known drugs used for the treatment of OA). In an aspect, the nanoscale diamond particles can be different types of functionalities (e.g., multi-modality nanoscale diamond particles) to achieve different purposes, such as those described above.

Carbon atoms in the nanoscale diamond particles are primarily sp³ hybridized. Carbons with sp² hybridized orbitals are considered defects in nanodiamond since all carbon atoms should have sp³ hybridized orbitals in a pure nanodiamond structure. The term “orbital hybridization”, as used herein, refers to the type of hybrid orbitals of the carbon atoms which make of the nanodiamond structure. In practice, X-ray diffraction (XRD) can be used to estimate or characterize the hybridization of carbon, where sp³ carbon has a diamond-like x-ray fingerprint and sp² carbon has a graphite-like x-ray fingerprint.

In one embodiment, the nanoscale diamond particles include carbon atoms, where the carbon atoms have an orbital hybridization that is about 50% to about 99% sp³ carbon, about 60% to about 99% sp³ carbon, about 70% to about 99% sp³ carbon, about 80% to about 99% sp³ carbon, about 90% to about 99% sp³ carbon, or about 95% to about 99% sp³ carbon.

Nanoscale diamond particles can be synthesized by a variety of processes, including detonation technique, laser ablation, high-energy ball milling of high-pressure high-temperature (HPHT) diamond microcrystals, plasma-assisted chemical vapor deposition (CVD), autoclave synthesis from supercritical fluids, chlorination of carbides, ion irradiation of graphite, electron irradiation of carbon ‘onions’, ultrasound cavitation, and a combination thereof.

In one or more aspects, nanoscale diamond particles are dissolved or dispersed in a biocompatible carrier fluid, which is selected from the group consisting of a simulated body fluid, a synovial fluid, a combination of both. One or more additional fluids can be added to the biocompatible carrier fluid to improve its functional property.

Nanoscale diamond particles on its own, as well as when dissolved or dispersed in a biocompatible carrier fluid, can be applied (e.g., coated onto, injected onto or into) in the artificial joint or natural joint.

The artificial joint is made of titanium-based alloys, ultra-high-molecular-weight polyethylene, high-density polyethylene, polytetrafluoroethylene, polyoxymethylene, stainless steel, cobalt-based alloys, chromium-based alloys, molybdenum-based alloys, or a combination thereof. In one or more aspects, nanoscale diamond particles can be used to lubricate artificial joints made of titanium-based alloys. Some of these alloys include Ti—Nb—Ta—Zr, Ti-6A-7Nb, Ti-6Al-4V, Ti-5Al-2.5Fe, or a combination thereof.

In one or more other aspects, the concentration of the nanoscale diamond particles distributed within biocompatible carrier fluid for injection is about 0.1 wt % to about 5 wt %, about 1 wt % to about 5 wt %, or about 2 wt % to about 5 wt % based upon a weight of the composition. In one or more other aspects, the concentration of the nanoscale diamond particles in the fluid operating within a joint (joint fluid) after an injection is about 0.001 wt % to about 0.25 wt %, 0.005 wt % to about 0.25 wt %, about 0.01 wt % to about 0.25 wt %, about 0.01 wt % to about 0.2 wt %, about 0.05 wt % to about 0.2 wt %, about 0.05 wt % to about 0.15 wt %, about 0.1 wt % to about 0.15 wt %, about 0.05 wt %, about 0.1 wt %, about 0.15 wt % or up to about 1 wt % based upon a weight of the composition.

In one or more other aspects, the term “natural joint” refers to hip joints, joints of hand, elbow joints, wrist joints, glenohumeral joint, acromioclavicular joint, sternoclavicular joints, vertebral articulations, temporomandibular joints, sacroiliac joints, knee joints, or articulations of foot. In one or more other aspects, the term “artificial joint” refers to implanted joints that replace hip joints, joints of hand, elbow joints, wrist joints, glenohumeral joint, acromioclavicular joint, sternoclavicular joints, vertebral articulations, temporomandibular joints, sacroiliac joints, knee joints, or articulations of foot.

In one or more aspects, intraarticular injections of nanoscale diamond particles comprising agents such as hyaluronic acid, hyaluronate salt, derivatives of hyaluronic acid, or other viscosupplement and other therapeutics can be applied to joints for mammals, for example for horses. More specifically they are preferably used in humans as well as for veterinary purposes. The agents can be linked to the nanoscale diamond particles and/or separately disposed within in the injection fluid.

A variety of methods for applying nanoscale diamond particles into the joint are provided. In one embodiment, the method includes coating an effective amount of nanoscale diamond particles onto the artificial joint prior to implant. In another embodiment, the method includes applying a composition disclosed herein into the joint during surgery to an artificial joint during implanting surgery. The composition comprises a biocompatible carrier fluid and an effective amount of nanoscale diamond particles dispersed in the biocompatible carrier fluid. In another embodiment, the method includes injecting (e.g., intra-articular injection) the composition disclosed herein into the artificial or natural joint.

### Liquid Formulations

Liquid formulations (e.g., the composition) contain an effective amount of nanoscale diamond particles, possibly with one or more additional active agents (e.g. hyaluronic acid, hyaluronate salt, derivatives of hyaluronic acid, or other viscosupplement or other therapeutics), dissolved or suspended in a biocompatible carrier fluid. Hyaluronic acid or other viscosupplement can be attached to NDs (covalently or non-covalently (e.g., via the functionality on the nanoscale diamond particles)) or be freely suspended along with NDs in a liquid carrier. Other therapeutics can be physically adsorbed to NDs or conjugated through chemical bonds. Other therapeutics can comprise anti-inflammatory drugs, antibacterial drugs, corticosteroids, chondroprotective drugs or other known drugs used for the treatment of OA.

Suitable carrier fluids include, but are not limited to, simulated body fluid, distilled water, de-ionized water, pure or ultrapure water, saline, and other physiologically acceptable aqueous solutions containing salts and/or buffers, such as phosphate buffered saline (PBS), Ringer's solution, and isotonic sodium chloride, or any other aqueous solution acceptable for administration to an animal or a human.

Preferably, liquid formulations are isotonic relative to physiological fluids and of approximately the same pH, ranging e.g., from about pH 4.0 to about pH 7.4, more preferably from about pH 6.0 to pH 7.0. The carrier fluid can include one or more physiologically compatible buffers, such as a phosphate buffers. One skilled in the art can readily determine a suitable saline content and pH for an aqueous solution for administration.

Liquid formulations may include one or more suspending agents, such as cellulose derivatives, sodium alginate, polyvinylpyrrolidone, gum tragacanth, or lecithin. Liquid formulations may also include one or more preservatives, such as ethyl or n-propyl p-hydroxybenzoate.

Liquid formulations may also contain minor amounts of polymers, surfactants, serum albumin, or other excipients well known to those of the art.

### Examples

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of aspects of the present disclosure.

To reproduce the joint environment in the body, tribological testing was performed in a simulated body fluid. SBF was prepared using a standard protocol. Sodium chloride, sodium bicarbonate, potassium chloride, potassium phosphate dibasic trihydrate, magnesium chloride hexahydrate, calcium chloride dihydrate, and sodium sulfate were dissolved in the right proportion in distilled water and held in an incubator at a constant temperature of 37° C. The solutions were used within a week to avoid any agglomeration and degradation of the mixture. The fresh solution was prepared for replicate tests.

Grade 5 purity (99.999% titanium) titanium balls (diameter 6 mm) and titanium flats (RMS roughness measured with Veeco Dektak 150 Surface Profiler Rq=50-60 nm) were used during testing. The hardness of the balls and flats measured with a Shimadzu Microhardness Tester were 4900 MPa and 3300 MPa, correspondingly. Tests were performed using the Anton Paar pin-on-disk macroscale tribometer in reciprocating mode. The length of the wear track was kept at 5 mm with reciprocal motion at 1 Hz. The samples were immersed in simulated body fluid during the tests and the temperature was kept at 37° C. to approximate conditions inside the body a closely as possible. During the tests, the applied load was varied from 0.25 N up to 1 N (maximum Hertzian contact pressure of 0.28-0.45 GPa). The contact pressures experienced by the surfaces during sliding were selected based on the previously reported contact pressure values for hip replacement contacts.

Small amounts of carboxylated detonation nanoscale diamond particles with 5 nm average volumetric size and zeta potential of −35 mV dispersed in DI water at 10 mg/mL were introduced directly to the simulated body fluid and the resulting solution was sonicated for at least 30 minutes. The added amounts of 1, 5, 10, 15, and 20 vol. % of the nanodiamond solution (NDS) correspond to 0.01, 0.05, 0.1, 0.15, and 0.2 wt % concentrations of nanoscale diamond particles in SBF, correspondingly. To confirm reproducibility of the results, at least three replicate tribotests were performed for each concentration of nanoscale diamond particles in SBF.

After tests, the simulated body fluid was removed and the samples were rinsed using DI water and the wear tracks were further analyzed. Optical images of the wear tracks were acquired using a Zeiss Optical Microscope. Raman analysis was performed Nicolet Almega XR Dispersive Raman spectrometer with 532 nm green laser. The samples were further characterized using an FEI Quanta 200 Scanning Electron Microscope (SEM) with energy dispersion x-ray analysis (EDX) to analyze the surface modification changes in the wear track.

To estimate the wear rate after the tests, the wear volume of the ball side is calculated as follows:

$$V = ( \frac{π h}{6} ) ( \frac{3 {d}^{2}}{4} + {h}^{2} ) \qquad ( 1 )$$

where d is the wear scar diameter, r is the radius of the ball, and h is the wear scar depth:

$$V = ( \frac{π h}{6} ) ( \frac{3 {d}^{2}}{4} + {h}^{2} ) \qquad ( 2 )$$

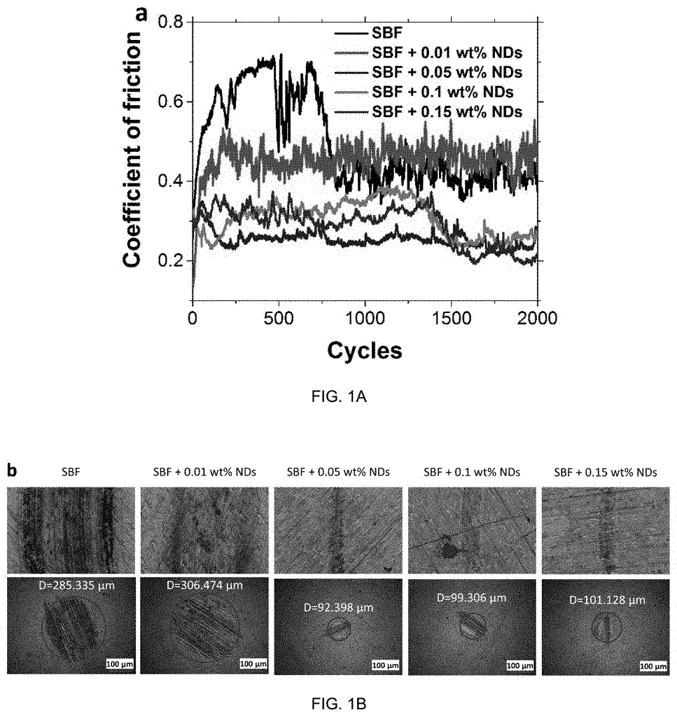

Changes in the coefficient of friction (COF) and wear values of the titanium surfaces were monitored during sliding under 0.25 N applied load. The results demonstrate that in contrast to pure SBF, the addition of 0.05 wt % of the nanodiamond leads to a two times reduction of the COF value. As demonstrated in FIG. 1A-B, 0.01 wt % shows almost negligible changes. Therefore, further analyses focused on 0.05, 0.1, and 0.15 wt. % concentrations.

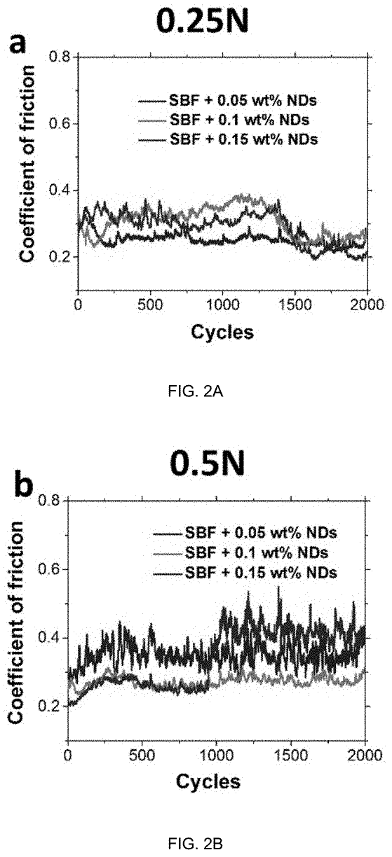

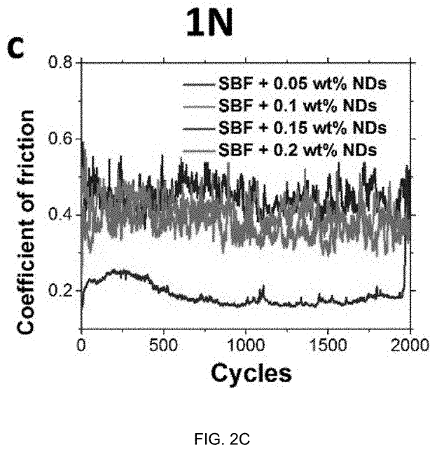

Increasing the applied load necessitates an increase in the amount of nanodiamond for enhanced performance. As demonstrated in FIG. 2, 0.1 wt. % works better at 0.5 N applied load, while 0.15 wt. % is optimal for 1 N applied load. This behavior is attributed to higher contact loads requiring a more uniform ND tribolayer, which may be achieved with higher ND content.

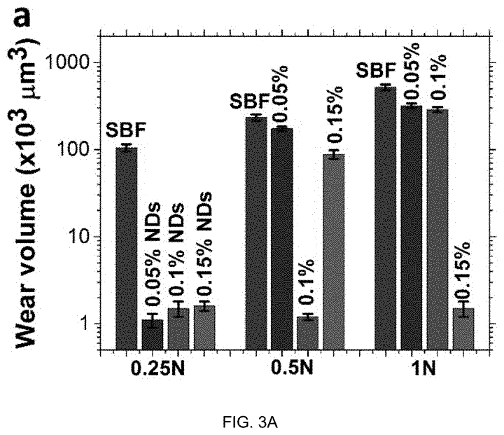

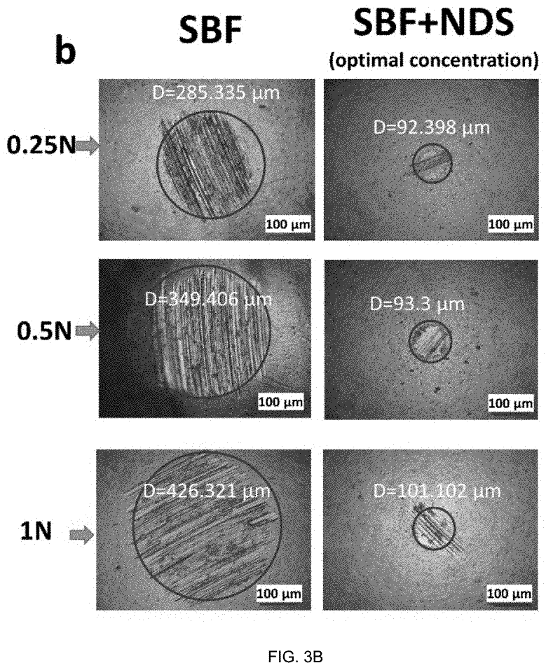

Analysis of the wear tracks further supports the benefits of different ND concentrations for each specific applied load (FIG. 3). Nevertheless, on average, the presence of NDs in SBF resulted in lower wear than for the SBF alone, and the resulting wear of the titanium surfaces depends on both parameters: ND concentration and applied load. FIG. 3 highlights the wear scar measurements for the optimal concentrations of NDs at each load.

Further understanding of the mechanism of lubrication improvement in the presence of NDs is possible through detailed characterization of the wear tracks corresponding to the tests in pure SBF and in SBF with an optimal concentration of NDs.

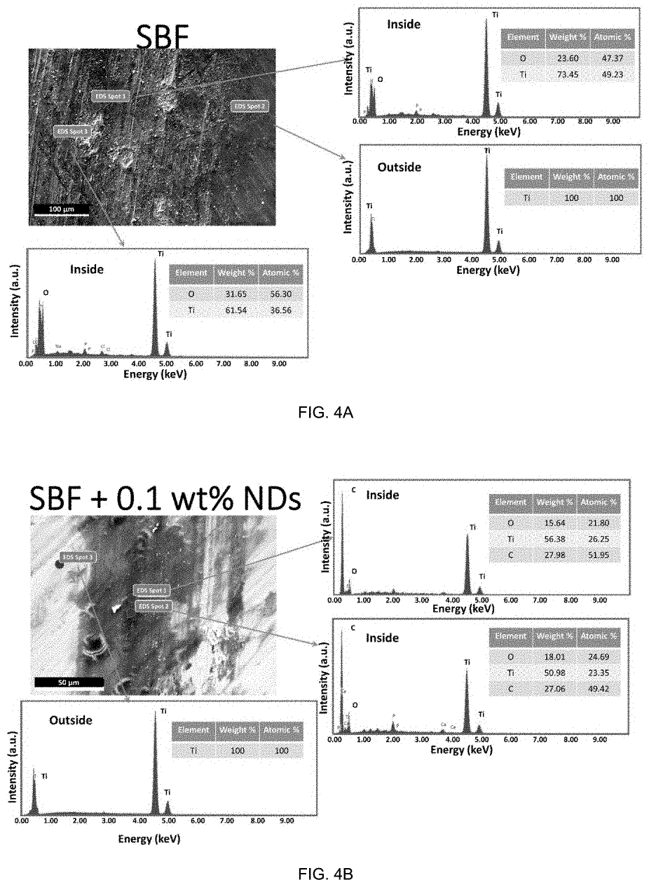

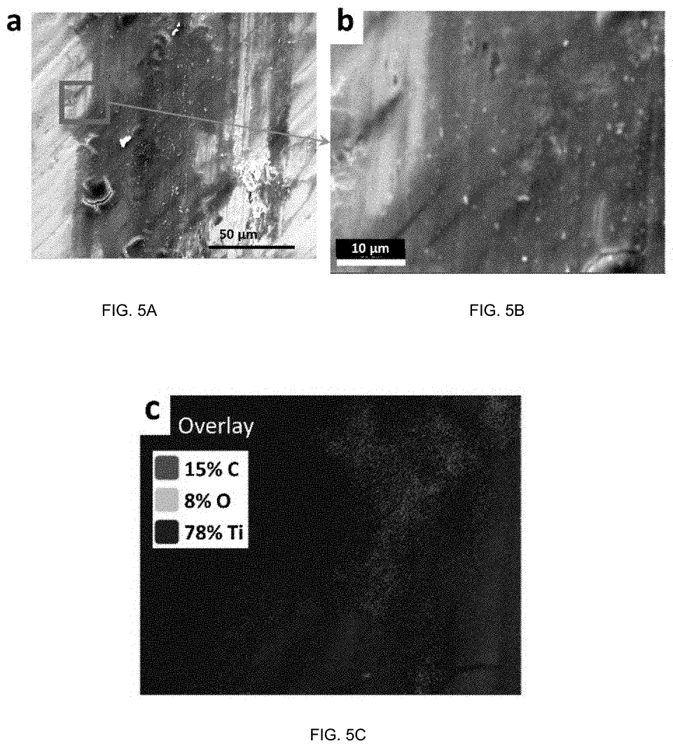

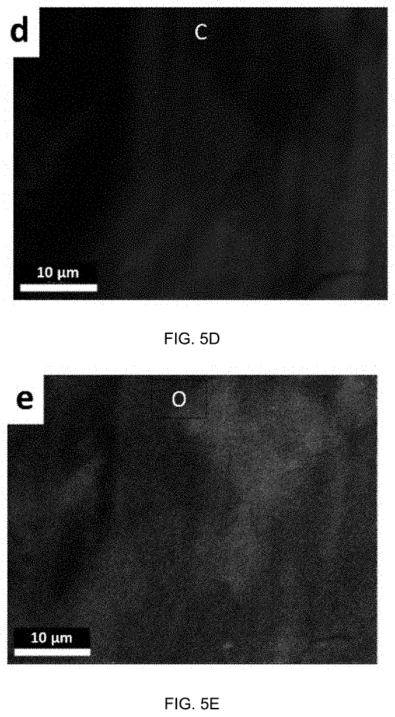

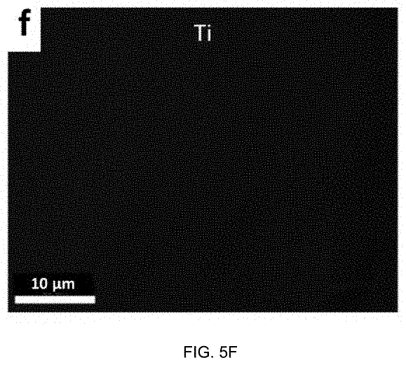

Analysis of the tracks formed during the sliding in pure SBF (FIG. 4) and SBF with 0.1 wt. % NDs at 0.5 N load (FIG. 5) was performed by acquiring energy dispersive x-ray spectroscopy (EDS) spectra from multiple points inside and outside of the wear tracks. The tests indicate that for the pure SBF, titanium oxide is dominant within the wear track. In the case of pure SBF, the titanium surfaces are exposed to sliding contact and their wear increases oxidation of the wear track region. When NDs are introduced to the SBF, the wear track oxidation is substantially lower. Interestingly, the EDS spectra inside the wear track clearly indicate the presence of carbon. Therefore, formation of the carbon rich layer in the wear track provides better tribological performance of the sliding system and protects the underlying titanium substrate from extensive wear. Similar wear and corrosion protection characteristics of carbon films were observed in case of graphitic layers. As a result, less titanium surface is exposed to the oxidation in SBF environment rich of corrosion promoting ions.

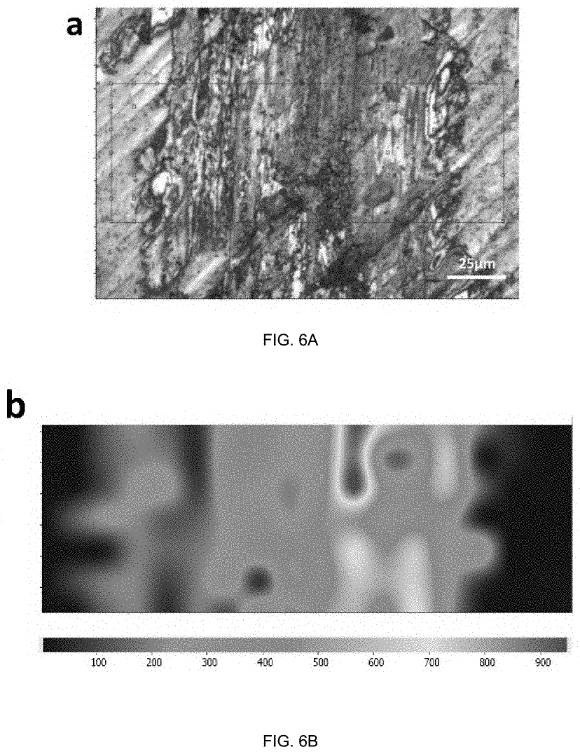

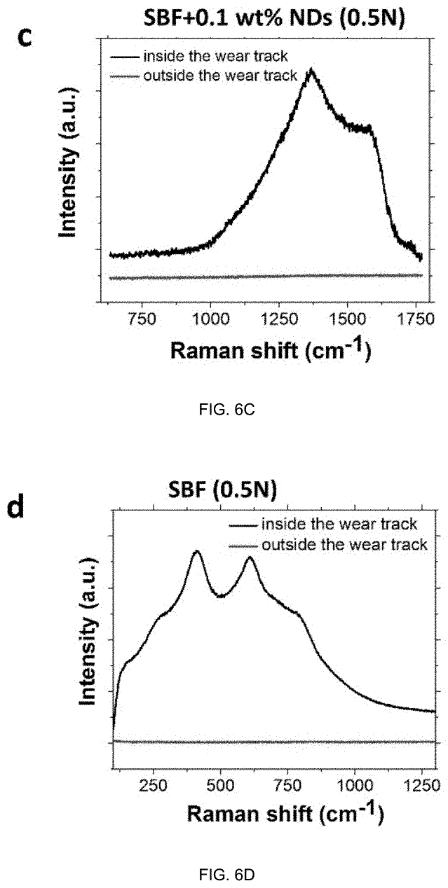

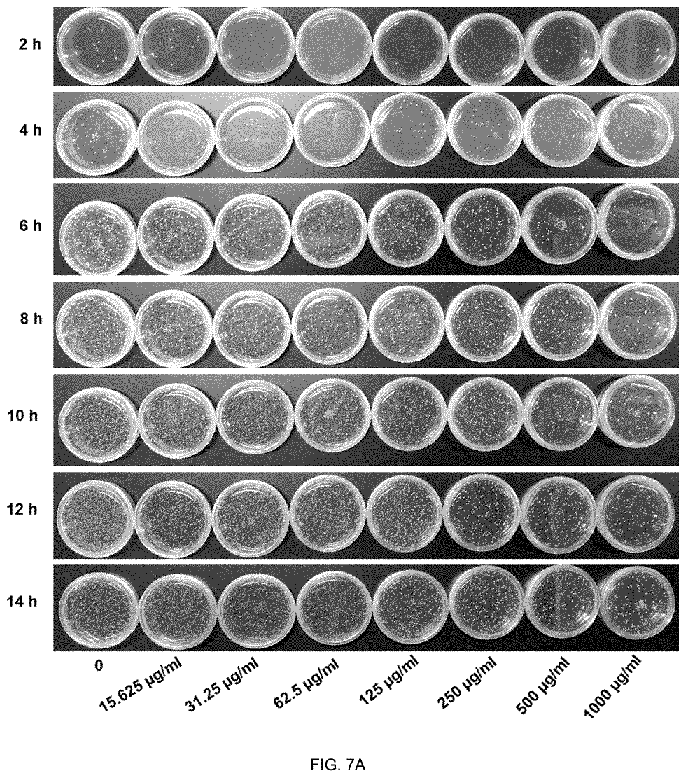

Further evaluation of the uniformity of the carbon coverage inside the wear track formed during sliding in the SBF+0.1 wt. % NDs is shown in FIGS. 6A-6D. A detailed EDS map of the SBF+0.1 wt. % NDs wear track (FIG. 6D) indicates uniformity of the carbon presence inside the wear track. Higher concentrations of oxygen correlates with regions of lower carbon presence. Thus, the presence of carbon plays a critical role in reducing oxidation and minimizing wear of the sliding surfaces.

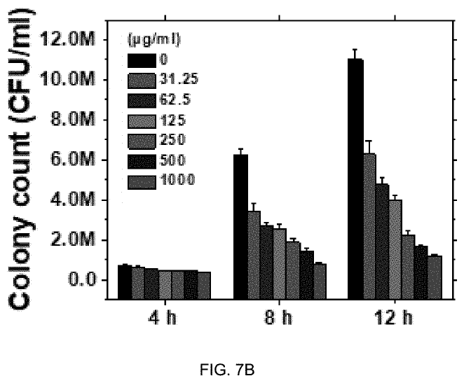

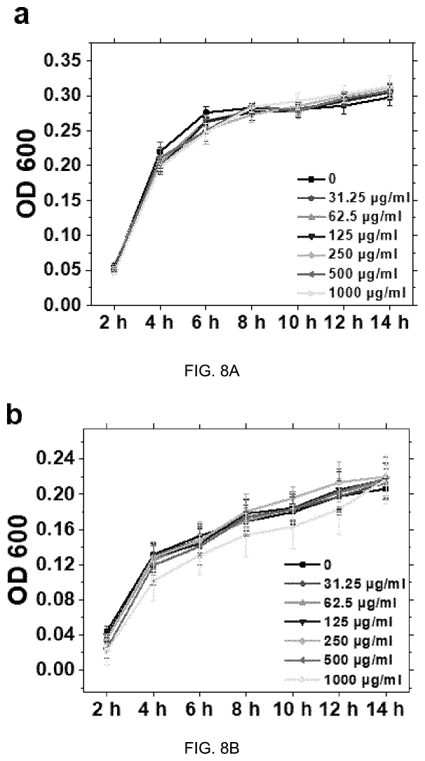

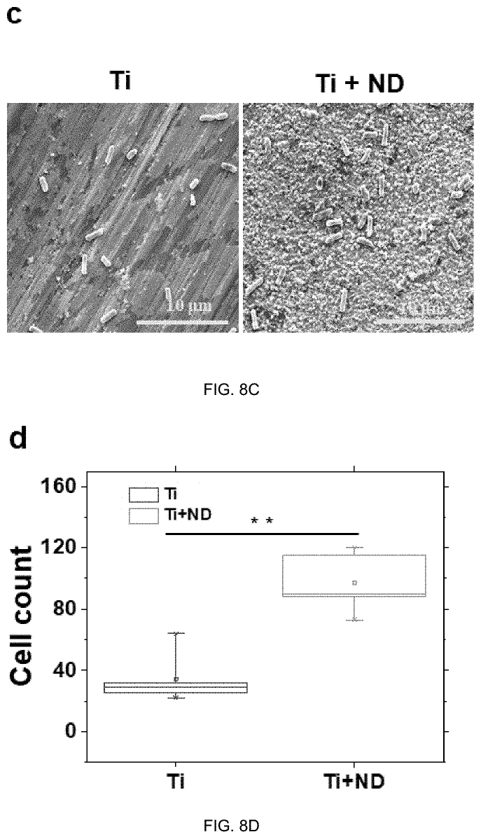

For S. aureus exposed to ND (FIG. 8A), the OD showed a concentration-dependent decrease. Consist with the result of the growth curve, the number of colonies was largely reduced compared with the control group (FIG. 7A-B). However, it was shown that the increase in OD of E. coli cultures exposed to ND was little influenced by a broad range of ND concentrations, in contrast to what has been observed for S. aureus. When exposed to ND-BA, the growth curve followed a similar trend for both S. aureus (FIG. 8B) and E. coli. To further investigate the antibacterial activity, SEM visualization conducted to evaluate the adhesion on Ti and Ti-UNCD plates. For S. aureus (FIG. 8C-D), a single layer of bacterial cells was generally observed, with a higher density of bacterial cells tightly settled on the Ti plate compared with Ti-UNCD plate, whereas much more bacterial cells attached on the Ti-UNCD plate for E. coli. Both visual observations and the quantitative bacterial cell count evaluation showed that the extent of bactericidal ability varied considerably among the tested microorganisms.

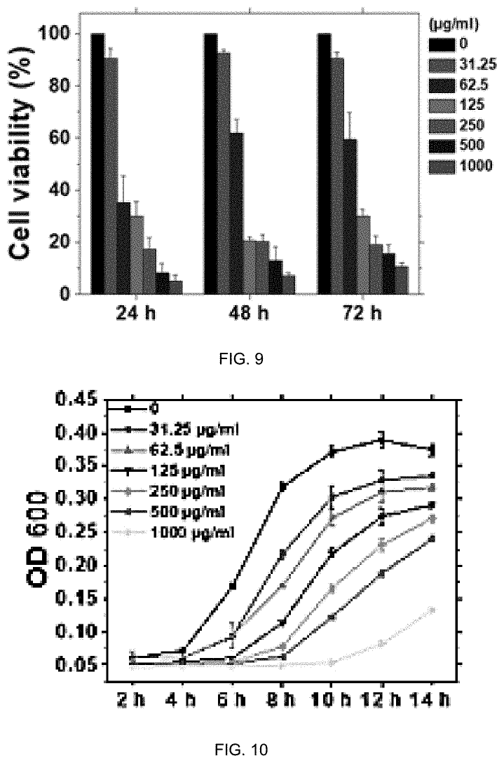

To investigate the influence of ND coating on cell morphology and cytoskeletal of MC3T3-E1 cells, fluorescence microscopy images were obtained. MTT was used to screen for any cytotoxic effect caused by ND/ND-PBS+10% Albumin (ND-BA). In case of ND, cells exhibited a significant reduction in viable cell numbers at a concentration of 62.5 μg/ml, whereas the cell cytotoxicity levels were not significantly different when treated with ND-BA even if at a higher concentration up to 1800 μg/ml (FIG. 9). It is obvious that ND-BA particles showed improved viability compared to ND.

For S. aureus exposed to ND (FIG. 10), the OD showed a concentration-dependent decrease. Consist with the result of the growth curve, the number of colonies was largely reduced compared with the control group.

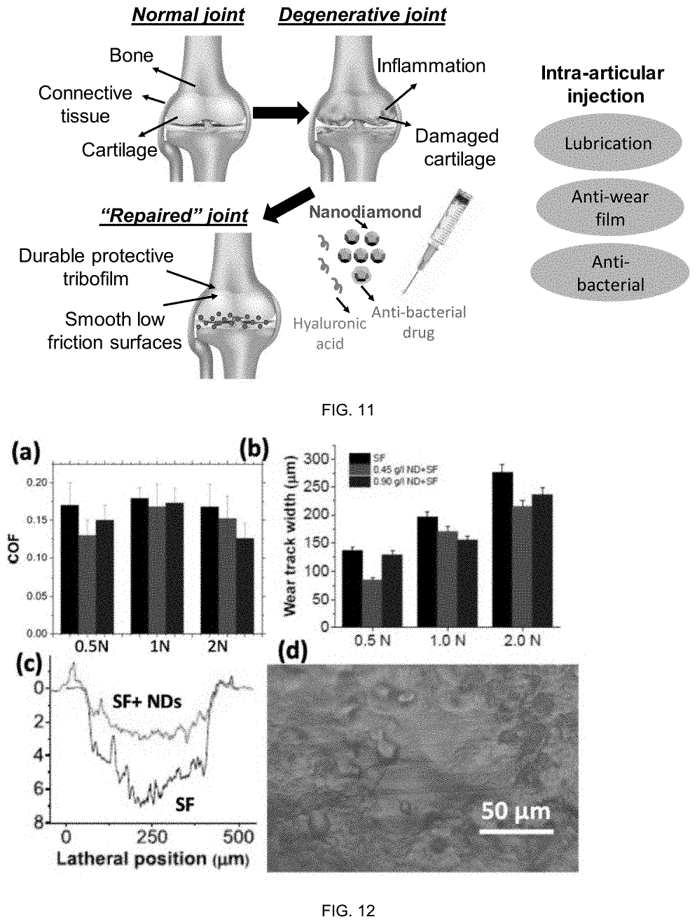

FIG. 11 illustrates schematics of a synergistic approach using NDs for treatment of early-stage OA and wear-induced damage in the joints.

In one embodiment, NDs intraarticular injections are aimed for supporting lubrication and reinforcing weakened natural cartilage in the early-stage of OA. Injection of saline-dispersed NDs into the synovial cavity will enhance lubrication between biological polymers (cartilage surfaces) and reinforce cartilage through the incorporation of NDs into the cartilage surface during joint movement and further distribution within the collagen matrix (FIG. 11). Increased stiffness of cartilage will further contribute to friction reduction. It is also expected that NDs can be embedded within the hyaluronic acid and lubricin molecular network formed on the cartilage surface (FIG. 11), reinforce it, and slow down the degradation of these lubricating macromolecules during sliding. It is also expected that the improved viscoelastic properties of the strengthened cartilage can reestablish the function of pressurizing interstitial fluid (i.e. fluid within the cartilage matrix) that supports contact loads. It is also possible that NDs can contribute to smoothening the cartilage surface from wear debris as it was observed in polymer and metallic surfaces, thus reducing the coefficient of friction and subsequent wear of the surfaces.

Particle size is a factor to maximize polymer reinforcement at minimum particle content. O ur estimates show that in order to achieve a 5× difference in stiffness of cartilage, <0.01 wt % of 4 nm NDs can be used. It is expected that the diffusion of 4 nm NDs particles or their small aggregates (for example, below about 50 nm) between collagen chains will be easier as compared to larger particles resulting in more deep penetration into cartilage and more uniform distribution. Larger aggregates (e.g. more than about 100 nm) can also compromise the microstructure of the collagen matrix. Therefore, prevention of NDs aggregation in high salt biological media is crucial and can be achieved through conjugation of NDs with branched polyglycerol (PG), which has been shown to provide excellent colloidal stability of NDs in saline and cell culture media. The PG coating also provides strong retention of hydration layers, contributing to enhanced lubricity. Due to the excellent chemical- and bio-resistance of NDs, they are not susceptible to chemical degradation, as opposed to many other reinforcing nanoparticles (e.g. nanosilica), providing long-term durability of the composites and eliminating the need for repeated injections after the strengthened cartilage is formed. Due to NDs exceptional biocompatibility, local administration, and the low concentration needed to achieve lubrication and mechanical enhancement, the proposed approach is expected to be non-toxic. While it is desirable that NDs stay within the cartilage and in the lubricating film and do not clear from the joint, however, consumption by macrophages and clearance through a lymphatic system is also possible.

NDs have been demonstrated as very efficient carriers of therapeutics and have shown markedly improved efficacy and safety for treatment in multiple preclinical studies. The loading capacity and binding strength of small molecules adsorbed/covalently grafted on NDs (FIG. 12) can be well-controlled through engineering of surface groups.

FIG. 12 illustrates graphs shown the friction (a) and wear (b,c) reduction in synovial fluid with NDs in contrast to pure SF in reciprocating sliding test on polymeric HDPE substrate at different ND dosing and load. No degradation of porcine cartilage lubricated with NDs in saline in the tribotest is observed (d). Friction analysis was performed using a macroscale pin-on-disk tribometer in reciprocating mode with titanium pin sliding against the porcine cartilage sample lubricated by synovial fluid with/without NDs. As shown in FIG. 12 (a-c), tribology tests (Ti ball on HDPE) for synovial fluid (SF) with/without NDs demonstrated both improved lubricity and reduced wear upon NDs addition at all loading parameters. NDs incorporation into a HDPE during sliding was confirmed using Raman analysis. A reduction in the coefficient of friction ˜10%, reduced (˜20%) and smoothened wear spot (FIG. 12d) were demonstrated in the porcine cartilage specimens lubricated with NDs (0.45 mg/ml in SF) as compared to pure SF in long-lasting tribotests (100,000 cycles, >10× longer than standard tribotests).

The present disclosure further includes the following embodiments.

1A. A composition for lubricating a joint (e.g., artificial or natural joint) in a subject (e.g., human) in need thereof; the composition comprising:
(i) a biocompatible carrier fluid; and
(ii) an effective amount of nanoscale diamond particles dispersed in the biocompatible carrier fluid to lubricate the joint when applied to the joint in the subject.
   2A. The composition according to paragraph 1A, wherein nanoscale diamond particles are spherical, elliptical, faceted, or a mixture thereof.
   3A. The composition according to any one of paragraphs 1A-2A, wherein the nanoscale diamond particles comprise carbon atoms, and
wherein the carbon atoms have an orbital hybridization that is about 50% to about 99% sp3 carbon, about 60% to about 99% sp3 carbon, about 70% to about 99% sp3 carbon, about 80% to about 99% sp3 carbon, about 90% to about 99% sp3 carbon, or about 95% to about 99% sp3 carbon.
   4A. The composition according to any one of paragraphs 1A-3A, wherein the nanoscale diamond particles are made by a process selected from the group consisting of detonation technique, laser ablation, high-energy ball milling of high-pressure high-temperature (HPHT) diamond microcrystals, plasma-assisted chemical vapor deposition (CVD), autoclave synthesis from supercritical fluids, chlorination of carbides, ion irradiation of graphite, electron irradiation of carbon ‘onions’, and ultrasound cavitation, and a combination thereof.
   5A. The composition according to any one of paragraphs 1A-4A, wherein the nanoscale diamond particles have a volumetric size of about 1 nm to about 20 nm, about 1 nm to about 10 nm, about 2 nm to about 10 nm, about 2 nm to about 8 nm, about 2 nm to about 6 nm, about 3 nm to about 5 nm, about 3 nm, about 4 nm, or about 5 nm.
   6A. The composition according to any one of paragraphs 1A-5A, wherein the nanoscale diamond particles are carboxylated.
   7A. The composition according to any one of paragraphs 1A-6A, wherein after an injection of the composition into a joint the nanoscale diamond particles are present in joint fluid at a concentration of about 0.001 wt % to about 0.25 wt %, 0.005 wt % to about 0.25 wt %, about 0.01 wt % to about 0.25 wt %, about 0.01 wt % to about 0.2 wt %, about 0.05 wt % to about 0.2 wt %, about 0.05 wt % to about 0.15 wt %, about 0.1 wt % to about 0.15 wt %, about 0.05 wt %, about 0.1 wt %, or about 0.15 wt % based upon an entire weight of the joint fluid.
   8A. The composition according to any one of paragraphs 1A-7A, wherein the biocompatible carrier fluid is selected from the group consisting of a saline, a simulated body fluid, a synovial fluid, a combination thereof, and mixtures thereof with one or more additional fluids.
   9A. The composition according to any one of paragraphs 1A-8A, wherein the composition is effective to reduce coefficient of friction at the joint by at least 20% (e.g., at least 60%, at least 75%, at least 90%, at least 95% (e.g., about 50 to 100%, about 50 to 90%, about 60 to 90%, about 70 to 90%, about 80 to 90%, about 80 to 95%)) as compared to the otherwise same composition except without the nanoscale diamond particles.
   10A. The composition according to any one of paragraphs 1A-9A, wherein the composition is effective to reduce wear at the joint by at least 20% (e.g., at least 60%, at least 75%, at least 90%, at least 95% (e.g., about 50 to 100%, about 50 to 90%, about 60 to 90%, about 70 to 90%, about 80 to 90%, about 80 to 95%)) as compared to the otherwise same composition except without the nanoscale diamond particles.
   11A. The composition according to any one of paragraphs 1A-10A, wherein the composition is effective to reduce bacteria growth by at least 20% (e.g., at least about 30%, at least about 45%, about least about 60%, at least about 75%, about least about 90% (e.g., about 20 to 90%, about 20 to 75%, about 45 to 90%, about 60 to 90%, about 75 to 90%)) as compared to the otherwise same composition except without the nanoscale diamond particles.
   12A. The composition according to any one of paragraphs 1A-11A, wherein the joint is the artificial joint and the artificial joint is made of a material selected from the group consisting of titanium-based alloys, ultra-high-molecular-weight polyethylene, high-density polyethylene, polytetrafluoroethylene, polyoxymethylene, stainless steel, cobalt-based alloys, chromium-based alloys, molybdenum-based alloys, and a combination thereof.
   13A. The composition according to any one of paragraphs 1A-12A, wherein the artificial joint is made of a material selected from the group consisting of Ti—Nb—Ta—Zr, Ti-6A-7Nb, Ti-6A-4V, Ti-5A-2.5Fe, and a combination thereof.
   14A. The composition according to any one of paragraphs 1A-13A, wherein the subject is a mammal.
   15A. The composition according to any one of paragraphs 1A-14A, wherein the joint is selected from the group consisting of hip joint, joint of hand, elbow joint, wrist joint, glenohumeral joint, acromioclavicular joint, sternoclavicular joint, vertebral articulation, temporomandibular joint, sacroiliac joint, knee joint, articulation of foot, and a combination thereof.
   16A. A method of lubricating a joint (e.g., an artificial joint or a natural joint) in a subject in need thereof, the method comprising: coating an effective amount of nanoscale diamond particles onto the joint to lubricate the joint, introducing an effective amount of a composition according to any one of paragraphs 1A-15A to the joint, and/or applying an effective amount of a composition according to any one of paragraphs 1A-15A to the joint.
   17A. The method according to paragraph 16A, wherein nanoscale diamond particles are spherical, elliptical, faceted, or a mixture thereof.
   18A. The method according to any one of paragraphs 16A-17A, wherein the nanoscale diamond particles comprise carbon atoms, and
wherein the carbon atoms have an orbital hybridization that is about 50% to about 99% sp3 carbon, about 60% to about 99% sp3 carbon, about 70% to about 99% sp3 carbon, about 80% to about 99% sp3 carbon, about 90% to about 99% sp3 carbon, or about 95% to about 99% sp3 carbon.
   19A. The method according to any one of paragraphs 16A-18A, wherein the nanoscale diamond particles are made by a process selected from the group consisting of detonation technique, laser ablation, high-energy ball milling of high-pressure high-temperature (HPHT) diamond microcrystals, plasma-assisted chemical vapor deposition (CVD), autoclave synthesis from supercritical fluids, chlorination of carbides, ion irradiation of graphite, electron irradiation of carbon ‘onions’, and ultrasound cavitation, and a combination thereof.
   20A. The method according to any one of paragraphs 16A-19A, wherein the nanoscale diamond particles have a volumetric size of about 1 nm to about 20 nm, about 1 nm to about 10 nm, about 2 nm to about 10 nm, about 2 nm to about 8 nm, about 2 nm to about 6 nm, about 3 nm to about 5 nm, about 3 nm, about 4 nm, or about 5 nm.
   21A. The method according to any one of paragraphs 16A-20A, wherein the nanoscale diamond particles are carboxylated.
   22A. The method according to any one of paragraphs 16A-21A, wherein the composition is effective to reduce coefficient of friction at the joint by at least 50% (e.g., at least 60%, at least 75%, at least 90%, at least 95% (e.g., about 50 to 100%, about 50 to 90%, about 60 to 90%, about 70 to 90%, about 80 to 90%, about 80 to 95%)) as compared to the otherwise same composition except without the nanoscale diamond particles.
   23A. The method according to any one of paragraphs 16A-22A, wherein the composition is effective to reduce wear at the joint by at least 20% (e.g., at least 60%, at least 75%, at least 90%, at least 95% (e.g., about 50 to 100%, about 50 to 90%, about 60 to 90%, about 70 to 90%, about 80 to 90%, about 80 to 95%)) as compared to the otherwise same composition except without the nanoscale diamond particles.
   24A. The method according to any one of paragraphs 16A-23A, wherein the composition is effective to reduce bacteria growth by at least 20% (e.g., at least about 30%, at least about 45%, about least about 60%, at least about 75%, about least about 90% (e.g., about 20 to 90%, about 20 to 75%, about 45 to 90%, about 60 to 90%, about 75 to 90%)) as compared to the otherwise same composition except without the nanoscale diamond particles.
   25A. The method according to any one of paragraphs 16A-24A, wherein the artificial joint is made of a material selected from the group consisting of titanium-based alloys, ultra-high-molecular-weight polyethylene, high-density polyethylene, polytetrafluoroethylene, polyoxymethylene, stainless steel, cobalt-based alloys, chromium-based alloys, molybdenum-based alloys, and a combination thereof.
   26A. The method according to any one of paragraphs 16A-25A, wherein the artificial joint is made of a material selected from the group consisting of Ti—Nb—Ta—Zr, Ti-6Al-7Nb, Ti-6Al-4V, Ti-5Al-2.5Fe, and a combination thereof.
   27A. The method according to any one of paragraphs 16A-26A, wherein the subject is a mammal.
   28A. The method according to any one of paragraphs 16A-27A, wherein the joint is selected from the group consisting of hip joint, joint of hand, elbow joint, wrist joint, glenohumeral joint, acromioclavicular joint, sternoclavicular joint, vertebral articulation, temporomandibular joint, sacroiliac joint, knee joint, articulation of foot, and a combination thereof.
   29A. The method according to any one of paragraphs 16A-28A, wherein the nanoscale diamond particles and/or the composition are applied (e.g., an intra-articular injection) to the joint prior to or at the same time as the artificial joint is implanted in the subject.
   30A. The method according to any one of paragraphs 16A-28A, wherein the nanoscale diamond particles and/or the composition are injected in the subject at or near the joint.
   31A. The composition according to any one of paragraphs 1A-15A or the method according to any one of paragraphs 16-30, wherein the nanoscale diamond particles have a zeta potential of about −20 mV to about −50 mV, about −25 mV to about −45 mV, or about −35 mV.
   32A. The composition according to any one of paragraphs 1A-15A or the method according to any one of paragraphs 16-30, wherein the nanoscale diamond particles can be in a form of tight aggregates with volumetric particle sizes between about 10 nm and about 50 nm.
   33A. The composition according to any one of paragraphs 1A-15A or the method according to any one of paragraphs 16-30, further comprising hyaluronic acid, hyaluronate salt, derivatives of hyaluronic acid, or other viscosupplement at the concentration of 0.01 wt % to 1 wt % when dispersed in the carrier fluid of the injectable composition.
   34A. The composition according to any one of paragraphs 1A-15A or the method according to any one of paragraphs 16-30, further comprising therapeutic for osteoarthritis treatment adsorbed on or conjugated with the nanoscale diamond particles.
   35A. The composition according to 43A, wherein the therapeutic comprises anti-inflammatory drugs, antibacterial drugs, corticosteroids, chondroprotective drugs or other known drugs used for the treatment of OA.
   36A. The composition according to any one of paragraphs 1A-15A or the method according to any one of paragraphs 16-30, wherein the nanoscale diamond particles are present in the biocompatible carrier fluid for injection at a concentration of about 0.01 wt % to about 5 wt %, or 0.1 wt % to about 1.0 wt %, based upon an entire weight of the composition.
   37A. The composition according to any one of paragraphs 1A-15A or the method according to any one of paragraphs 16-30, wherein the nanoscale diamond particles are hydroxilated, aminated, fluorinated, hydrogenated, conjugated with silane, acrylic groups, aliphatic chains polyethylene glycol.
   38A. The composition according to any one of paragraphs 1A-15A or the method according to any one of paragraphs 16-30, wherein the nanoscale diamond particles are conjugated with poly(glycerol) by ring opening polymerization in neat glycidol to introduce colloidal stability in biological media followed by conjugation to therapeutic molecules by carbodiimide or related activation.
   1B. A method of lubricating a joint in a subject in need thereof, the method comprising: introducing an effective amount of a composition into the joint to lubricate the joint, wherein the joint is an artificial joint or a natural joint, wherein the composition comprises (i) a biocompatible carrier fluid; and (ii) an effective amount of nanoscale diamond particles dispersed in the biocompatible carrier fluid to lubricate the joint when applied to the joint in the subject.
   2B. The method of paragraph 1, wherein introducing includes injecting the composition into the joint.
   3B. The method of any of the preceding paragraphs, wherein injecting includes an intra-articular injection.
   4B. The method of any of the preceding paragraphs, wherein the intra-articular injection includes intra-articular injection into the joint is selected from the group consisting of: hip joint, joint of hand, elbow joint, wrist joint, glenohumeral joint, acromioclavicular joint, sternoclavicular joint, vertebral articulation, temporomandibular joint, sacroiliac joint, knee joint, and articulation of foot.
   5B. The method of any of the preceding paragraphs, wherein the subject is a human.
   6B. The method of any of the preceding paragraphs, wherein the joint in an artificial joint or a natural joint.
   7B. The method of any of the preceding paragraphs, wherein the nanoscale diamond particles are spherical, elliptical, faceted, or a mixture thereof.
   8B. The method of any of the preceding paragraphs, wherein the nanoscale diamond particles comprise carbon atoms, and wherein the carbon atoms have an orbital hybridization that is about 80% to about 99% sp³ carbon.
   9B. The method of any of the preceding paragraphs, wherein the nanoscale diamond particles and spherical and have a volumetric size of about 2 nm to about 6 nm.
   10B. The method of any of the preceding paragraphs, wherein the nanoscale diamond particles are carboxylated.
   11B. The method of any of the preceding paragraphs, wherein the nanoscale diamond particles are present in the biocompatible carrier fluid for injection at a concentration of about 0.01 wt % to about 5 wt %, or 0.1 wt % to about 1.0 wt %, based upon an entire weight of the composition.
   12B. The method of any of the preceding paragraphs, wherein the biocompatible carrier fluid is selected from the group consisting of a simulated body fluid, a synovial fluid, a combination thereof, and mixtures thereof with one or more additional fluids.
   13B. The method of any of the preceding paragraphs, wherein the composition is effective to reduce coefficient of friction at the joint by at least 50% as compared to the otherwise same composition except without the nanoscale diamond particles.
   14B. The method of any of the preceding paragraphs, wherein the composition is effective to reduce wear at the joint by at least one order of magnitude as compared to the otherwise same composition except without the nanoscale diamond particles.
   15B. The method of any of the preceding paragraphs, wherein the composition is effective to reduce bacteria growth by at least 20% as compared to the otherwise same composition except without the nanoscale diamond particles.
   16B. The method of any of the preceding paragraphs, wherein the joint is the artificial joint and the artificial joint is made of a material selected from the group consisting of titanium-based alloys, ultra-high-molecular-weight polyethylene, high-density polyethylene, polytetrafluoroethylene, polyoxymethylene, stainless steel, cobalt-based alloys, chromium-based alloys, molybdenum-based alloys, and a combination thereof.
   17B. The method of any of the preceding paragraphs, wherein the artificial joint is made of a material selected from the group consisting of Ti—Nb—Ta—Zr, Ti-6A-7Nb, Ti-6A-4V, Ti-5A-2.5Fe, and a combination thereof.
   18B. A method of lubricating a joint in a human, the method comprising: injecting an effective amount of a composition into the joint to lubricate the joint, wherein the joint is a natural joint, wherein the composition comprises (i) a biocompatible carrier fluid that is selected from the group consisting of a simulated body fluid, a synovial fluid, a combination thereof, and mixtures thereof with one or more additional fluids and species; and (ii) an effective amount of nanoscale diamond particles dispersed in the biocompatible carrier fluid to lubricate the joint when applied to the joint in the subject, wherein the nanoscale diamond particles are present at a concentration of about 0.01 wt % to about 5 wt % in the biocompatible carrier fluid based upon an entire weight of the composition, wherein the nanoscale diamond particles comprise carbon atoms, and wherein the carbon atoms have an orbital hybridization that is about 80% to about 99% sp³ carbon, and wherein the nanoscale diamond particles and spherical and have a volumetric size of about 2 nm to about 6 nm.
   19B. The method of paragraph 18, wherein injecting includes an intra-articular injection.
   20B. The method of paragraph 18 or 19, wherein the intra-articular injection includes intra-articular injection into the joint is selected from the group consisting of: hip joint, joint of hand, elbow joint, wrist joint, glenohumeral joint, acromioclavicular joint, sternoclavicular joint, vertebral articulation, temporomandibular joint, sacroiliac joint, knee joint, and articulation of foot.
   21B. The method according to any one of paragraphs 1BA-20B, wherein the nanoscale diamond particles can be in a form of tight aggregates with volumetric particle sizes between about 10 nm and about 50 nm.
   22B. The method according to any one of paragraphs 1BA-20B, further comprising hyaluronic acid, hyaluronate salt, derivatives of HA, or other viscosupplement at the concentration of 0.01 wt % to 1 wt % when dispersed in the carrier fluid of the injectable composition.
   23B. The method according to any one of paragraphs 1BA-20B, further comprising therapeutic for osteoarthritis treatment adsorbed on or conjugated with the nanoscale diamond particles.
   24B. The composition according to 23B, wherein the therapeutic comprises anti-inflammatory drugs, antibacterial drugs, corticosteroids, chondroprotective drugs or other known drugs used for the treatment of OA.
   25B. The method according to any one of paragraphs 1BA-20B, wherein the nanoscale diamond particles are present in the biocompatible carrier fluid for injection at a concentration of about 0.01 wt % to about 5 wt %, or 0.1 wt % to about 1.0 wt %, based upon an entire weight of the composition.
   26B. The method according to any one of paragraphs 1BA-20B, wherein the nanoscale diamond particles are hydroxilated, aminated, fluorinated, hydrogenated, conjugated with silane, acrylic groups, aliphatic chains polyethylene glycol.
   27B. The method according to any one of paragraphs 1BA-20B, wherein the nanoscale diamond particles are conjugated with poly(glycerol) by ring opening polymerization in neat glycidol to introduce colloidal stability in biological media followed by conjugation to therapeutic molecules by carbodiimide or related activation.

It should be emphasized that the above-described aspects of the present disclosure are merely possible examples of implementations, and are set forth only for a clear understanding of the principles of the disclosure. Many variations and modifications may be made to the above-described aspects of the disclosure without departing substantially from the spirit and principles of the disclosure. All such modifications and variations are intended to be included herein within the scope of this disclosure.

What is claimed is:

1. A method of lubricating a joint in a subject in need thereof, the method comprising: introducing an effective amount of a composition into the joint to lubricate the joint, wherein the joint is an artificial joint or a natural joint, wherein the composition comprises (i) a biocompatible carrier fluid; and (ii) an effective amount of nanoscale diamond particles dispersed in the biocompatible carrier fluid to lubricate the joint when applied to the joint in the subject.

2. The method of claim 1, wherein introducing includes injecting the composition into the joint.

3. The method of claim 2, wherein injecting includes an intra-articular injection.

4. The method of claim 3, wherein the intra-articular injection includes intra-articular injection into the joint is selected from the group consisting of: hip joint, joint of hand, elbow joint, wrist joint, glenohumeral joint, acromioclavicular joint, sternoclavicular joint, vertebral articulation, temporomandibular joint, sacroiliac joint, knee joint, and articulation of foot.

5. The method of claim 4, wherein the subject is a human.

6. The method of claim 5, wherein the joint in an artificial joint or a natural joint.

7. The method of claim 1, wherein the nanoscale diamond particles are spherical, elliptical, faceted, or a mixture thereof.

8. The method of claim 1, wherein the nanoscale diamond particles comprise carbon atoms, and wherein the carbon atoms have an orbital hybridization that is about 80% to about 99% sp³ carbon.

9. The method of claim 1, wherein the nanoscale diamond particles and spherical and have a volumetric size of about 2 nm to about 6 nm.

10. The method of claim 1, wherein the nanoscale diamond particles are carboxylated.

11. The method of claim 1, wherein the nanoscale diamond particles are present in the biocompatible carrier fluid at a concentration of about 0.01 wt % to about 5 wt %, or 0.1 wt % to about 1.0 wt %, based upon an entire weight of the composition.

12. The method of claim 1, wherein the biocompatible carrier fluid is selected from the group consisting of a saline, simulated body fluid, a synovial fluid, a combination thereof, and mixtures thereof with one or more additional fluids.

13. The method of claim 1, wherein the composition is effective to reduce coefficient of friction at the joint by at least 20% as compared to the otherwise same composition except without the nanoscale diamond particles.

14. The method of claim 1, wherein the composition is effective to reduce wear at the joint by at least 20% as compared to the otherwise same composition except without the nanoscale diamond particles.

15. The method of claim 1, wherein the composition is effective to reduce bacteria growth by at least 20% as compared to the otherwise same composition except without the nanoscale diamond particles.

16. The method of claim 1, wherein the joint is the artificial joint and the artificial joint is made of a material selected from the group consisting of titanium-based alloys, ultra-high-molecular-weight polyethylene, high-density polyethylene, polytetrafluoroethylene, polyoxymethylene, stainless steel, cobalt-based alloys, chromium-based alloys, molybdenum-based alloys, and a combination thereof.

17. The method of claim 1, wherein the artificial joint is made of a material selected from the group consisting of Ti—Nb—Ta—Zr, Ti-6Al-7Nb, Ti-6A-4V, Ti-5A-2.5Fe, and a combination thereof.

18. The method according to claim 1, further comprising hyaluronic acid, hyaluronate salt, derivative of hyaluronic, or a viscosupplement at the concentration of 0.01 wt % to 1 wt % when dispersed in the carrier fluid of the injectable composition.

19. The method according to claim 1, further comprising therapeutic for osteoarthritis treatment adsorbed on or conjugated with the nanoscale diamond particles, optionally, wherein the therapeutic comprises anti-inflammatory drugs, antibacterial drugs, corticosteroids, chondroprotective drugs or other known drugs used for the treatment of OA.

20. The method according to claim 1, wherein the nanoscale diamond particles are conjugated with poly(glycerol) to introduce colloidal stability in biological media.
